(19) United States
(12) Patent Application Publication
(10) Pub. No.: US 2003/0236491 A1
(43) Pub. Date: Dec. 25, 2003

(54) Apparatus for extracting milk from lactating women

(75) Inventors: Bruce McKendry, Benecia, CA (US); Sung Lee, Elk Grove, CA (US); Wallace Charles Seaman, Cool, CA (US)

Correspondence Address: GREER, BURNS & CRAIN, LTD., Suite 2500, 300 South Wacker Drive, Chicago, IL, 60606, US

(73) Assignee: L. JASON CLUTE

(21) Appl. No.: 10/179,591

(22) Filed: Jun. 24, 2002

(51) Int. Cl. A61M 1/06

(52) U.S. Cl. 604/74

(57) ABSTRACT

Milk is extracted from a human breast by placing the breast in a cup having a vacuum port. The cup also has a flexible liner that allows vacuum to extract milk when the liner is in a first, open position, the milk being drawn into the vacuum port by the vacuum when the liner is in the first position. The liner also has a second position in which the nipple of the breast is compressed enough to substantially reduce the flow of milk from the breast. The liner is opened and closed in a pulsating manner at a predetermined pulsation rate. The user may select a pulsation ratio that produces acceptable milk transfer and comfort. Optimum pulsation ratios vary from user to user, but are generally between about 20% and about 80% for the vacuum portion of the pulsation cycle.

### FIELD OF THE INVENTION

This invention relates to apparatus for extracting milk from lactating women, and more particularly, to breast pumps that use pulsation within a range of pulsation ratios to enhance comfort and milk production.

### BACKGROUND OF THE INVENTION

Mechanical breast pumps for lactating women apply a vacuum to the breast, which draws milk from the nipple and collects the milk in a vessel such as a bottle. Most conventional breast pumps apply an oscillating vacuum to the breast from a single chamber in a breast cup, but Whittlestone U.S. Pat. No. 4,607,596 discloses a device with dual chambers that uses a liner in the breast cup and applies pulsation to the outside of the liner at regular intervals. This pulsation reduces congestion at the breast when the liner is closed, giving greater relief from the application of vacuum.

While the Whittlestone patent suggests a pulsation rate of 40 pulsations per minute, it does not disclose a suitable pulsation ratio or time within a pulsation cycle during which the vacuum should be applied to the breast to accomplish milk production. The Whittlestone patent also does not recognize that optimum milk production is achieved at different pulsation ratios for different women, and even for one woman at different times.

Accordingly, one object of this invention is to provide a new and improved apparatus for extracting milk from lactating women.

Another object is providing new and improved breast pumps that use pulsation within a range of pulsation ratios to enhance comfort and milk production.

Still another object is to provide new and improved breast pumps having a pulsation ratio that can be adjusted by the user.

### SUMMARY OF THE INVENTION

In keeping with one aspect of this invention, milk is extracted from a lactating human breast by placing the breast in a cup. A vacuum source is connected to the cup. The cup has a flexible liner that allows milk to be extracted when the liner is in the first, open position. The milk is drawn into the breast cup by the vacuum and conveyed into a collecting bottle when the liner is in the first position. The liner also has a second or closed position in which the nipple is compressed enough to substantially reduce the flow of milk from the breast. The liner is closed and opened in a pulsating manner at a predetermined pulsation rate that produces acceptable milk transfer and comfort. The relationship of time between the open and closed positions can be expressed as a ratio. Such a pulsation ratio may vary within and among users, but is between about 20% and about 80% open of the total of each pulsation cycle (i.e., time open/total time of entire cycle).

In another aspect of the invention, the pulsation ratio is variable, and is adjustable by the user.

### BRIEF DESCRIPTION OF THE DRAWINGS

The above mentioned and other features of this invention and the manner of obtaining them will become more apparent, and the invention itself will be best understood by reference to the following description of an embodiment of the invention taken in conjunction with the accompanying drawings, in which:

FIG. 1 is a side cross-sectional view of a milk collection assembly that can be used in the present invention;

FIG. 2 is a top cross-sectional view of the milk collection assembly of FIG. 1, taken along lines 2-2 in FIG. 1;

FIG. 3 is a cross-sectional view of the breast cup in the assembly of FIG. 1, shown with the liner partially collapsed by vacuum;

FIG. 4 is an exploded view of the breast cup of FIG. 1;

FIG. 5 is a block diagram of part of one embodiment of the present invention, using the milk collecting assembly of FIG. 1;

FIG. 6 is a block diagram of a control system for use with the apparatus of FIG. 5; and

FIG. 7 is a graph comparing the expected relationship between pulsation ratio and milk transfer, with the relationship obtained with the present invention.

### DETAILED DESCRIPTION

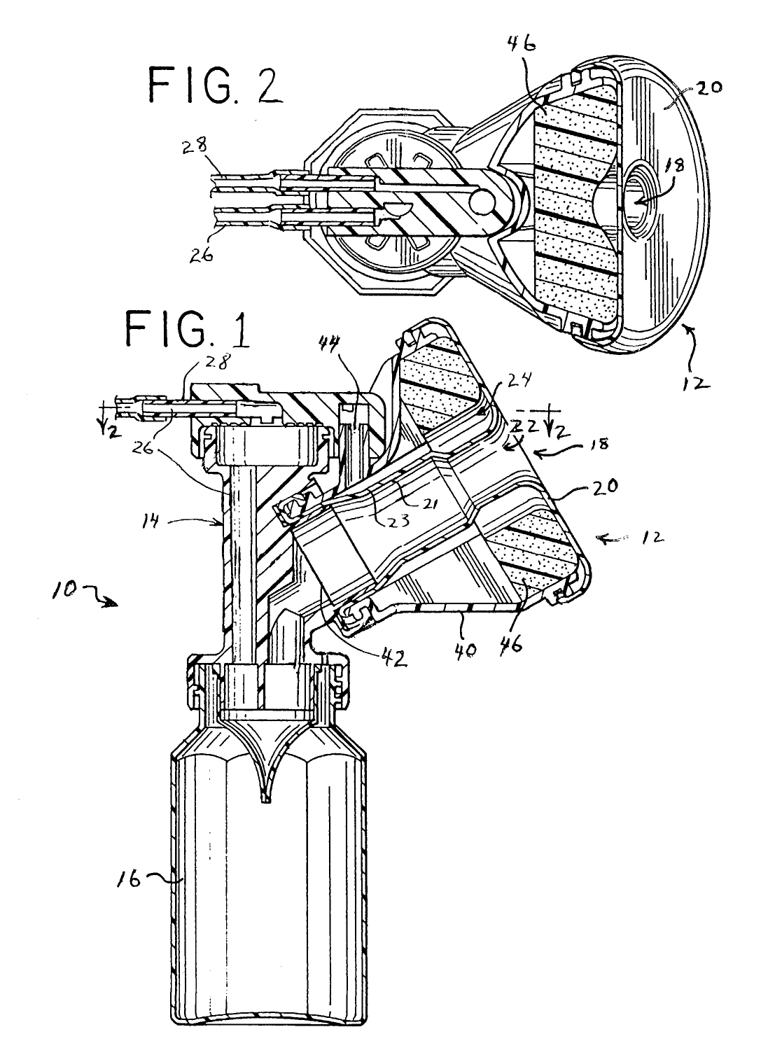

As seen in FIGS. 1 and 2, a milk-collecting unit 10 includes a breast cup assembly 12, a manifold assembly 14 and a milk collection unit 16. The cup assembly 12 includes an opening 18 into which a lactating human breast may be inserted for extraction of milk.

The cup assembly 12 also has a flexible liner 20 that extends within the cup assembly 12. The liner 20 can be made of silicon or other suitable material. The inside 21 of the liner 20 forms part of an inner chamber 22 around the opening 18, and the outside 23 of the liner 20 forms part of an outer chamber 24.

Manifold 14 includes a vacuum tube 26 and a pulse tube 28, as seen in FIG. 2. The vacuum tube 26 passes vacuum into the manifold assembly 14, the container 16 and the vacuum chamber 22 (FIG. 1). In use, the breast seals the opening 18 so that when the liner 20 is pressed against the breast at the opening 18, milk is drawn into the container 16 by the vacuum, through the core of the liner 22 and an optional duck bill valve. When vacuum is applied to the outer chamber 24, the liner 20 moves to the first, or open position in FIG. 1.

A vacuum can be applied to the outer chamber 24, or the outer chamber 24 can be raised to atmospheric pressure, although higher pressures could be used, if desired. When air is admitted through the pulsation tube 28, atmospheric air fills the second chamber 24, and the liner 20 closes in the manner shown in FIG. 3. The liner closes (second position) due to the differential pressures across the walls 21, 23 of the liner between the two chambers. The vacuum chamber 22 draws the liner 20 gently against the breast, and into contact with exposed milk channel openings (nipple sinuses), the contact, or compressive load substantially reducing milk flow from the nipple. Reducing the milk channel in this manner reduces vacuum exposure (hence, milk flow) to the breast and supports the breast in a gentle, compressive force mode. This compressive force tends to relieve congestion of blood, lymph, and other body fluids brought to the front portion of the breast tissue by the vacuum applied when the liner is in the first position.

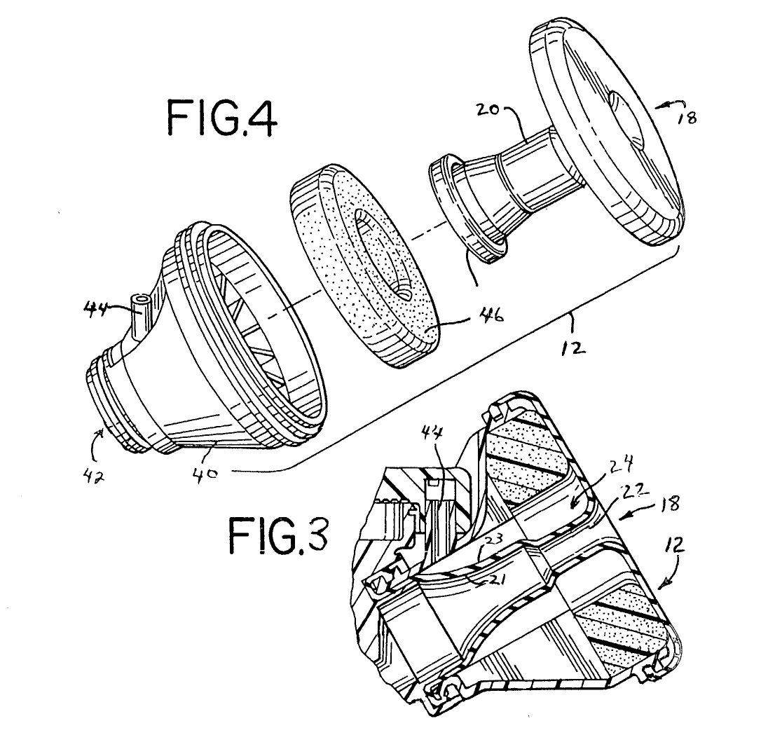

FIG. 4 is an exploded view of the cup assembly of FIG. 1. The cup assembly includes a case or housing 40 having a vacuum port 42 and a pulsed air port 44. The pulsation tube 28 of FIG. 1 communicates with the port 42 in use. The liner 20 can wrap around over an insert 46 and the housing 40 on one end, and around the vacuum port 42 on the other end of the housing. Thus, the liner 20 and the housing 40 form the outer chamber 24 (FIG. 3) through which pulsed air and vacuum are alternately applied, and the liner 20 forms the chamber 22, which is sealed by the breast.

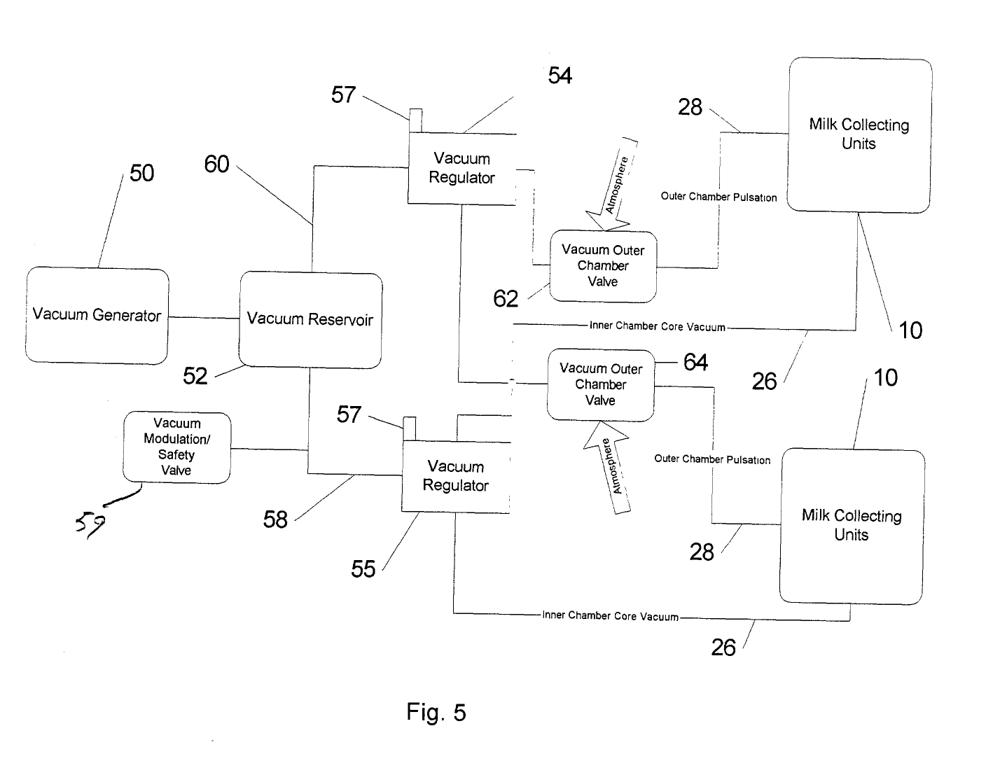

The vacuum and pulsed pressure in the airlines 26 and 28, respectively, can be produced in various ways, including the manner shown in FIG. 5. A vacuum generator 50 produces vacuum in an optional reservoir 52, preferably at about three to ten inches of mercury (Hg). The vacuum generator 50 could be a rotary vane pump, a scroll type pump, a piston-type pump, a Woble piston type pump, a diaphragm pump, a linear pump, a bellow, or other vacuum generator.

Desired vacuum levels can be established with the assistance of vacuum regulators 54, 55 having mechanical or other adjustments 57. A vacuum line 58 can be provided to operate the two breast cup assemblies 10 through the regulator 55 and the vacuum lines 26. The vacuum is preferably modulated, but a constant vacuum is also suitable for extracting milk in accordance with the present invention. Modulation can be accomplished with a vacuum modulation/safety valve 59, which can be a mechanical vacuum release valve, for example.

An airline 60 provides vacuum to valves 62, 64. The valves 62, 64 in turn, selectively provide alternating vacuum and atmospheric air to the pulsed airlines 28 in the breast cup assemblies 10. A pressure differential of about 0.5 to 2.0 Hg is suitable to open and close the liner. The liner wall movement (pulsation) is produced using pulsator valves 62,64, which use vacuum in the optional reservoir 52 to open the liner 20 and admit atmospheric air to close the liner, in conjunction with the vacuum present from the vacuum port 42.

Pulsator valves 62, 64 have two functions: first, they allow vacuum from the reservoir 52 (if used) into pulsation tubes 28, which are linked to the breast cup; which opens the liner 20; second, they allow atmospheric pressure into these same lines (and the cups) to allow system vacuum to close the liner 20. Valves made by BioChem Valve, Inc. in Oakland, Calif., Part No. 0075T3, S119, 12 vol., D.C., called a three-way solenoid valve having an “open,” “close” and “open to atmosphere” positions is one example of a suitable valve.

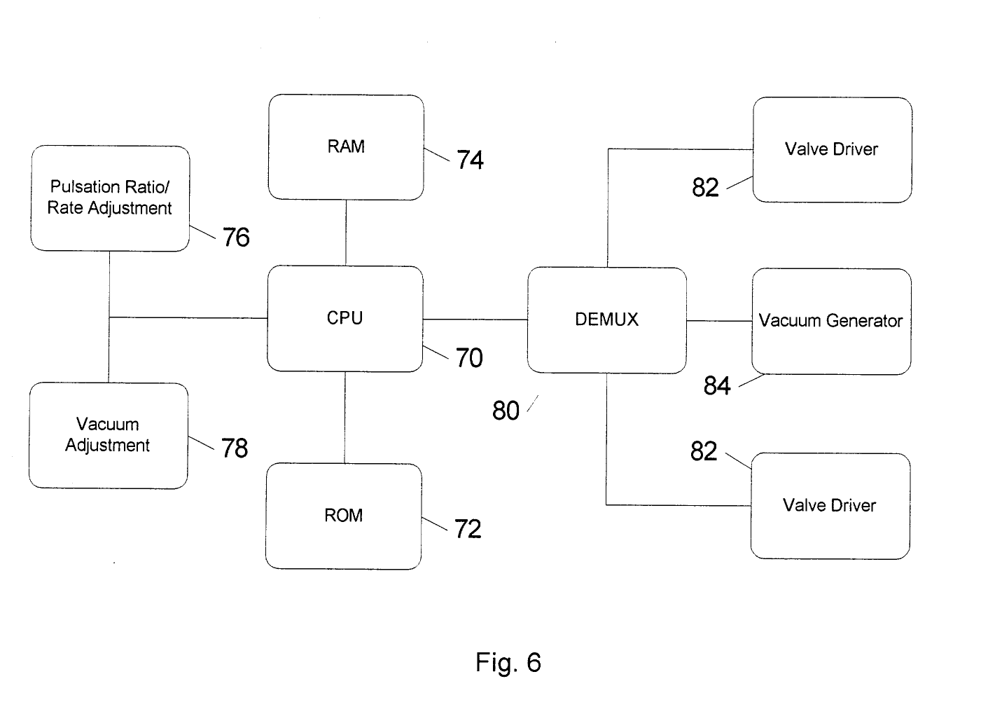

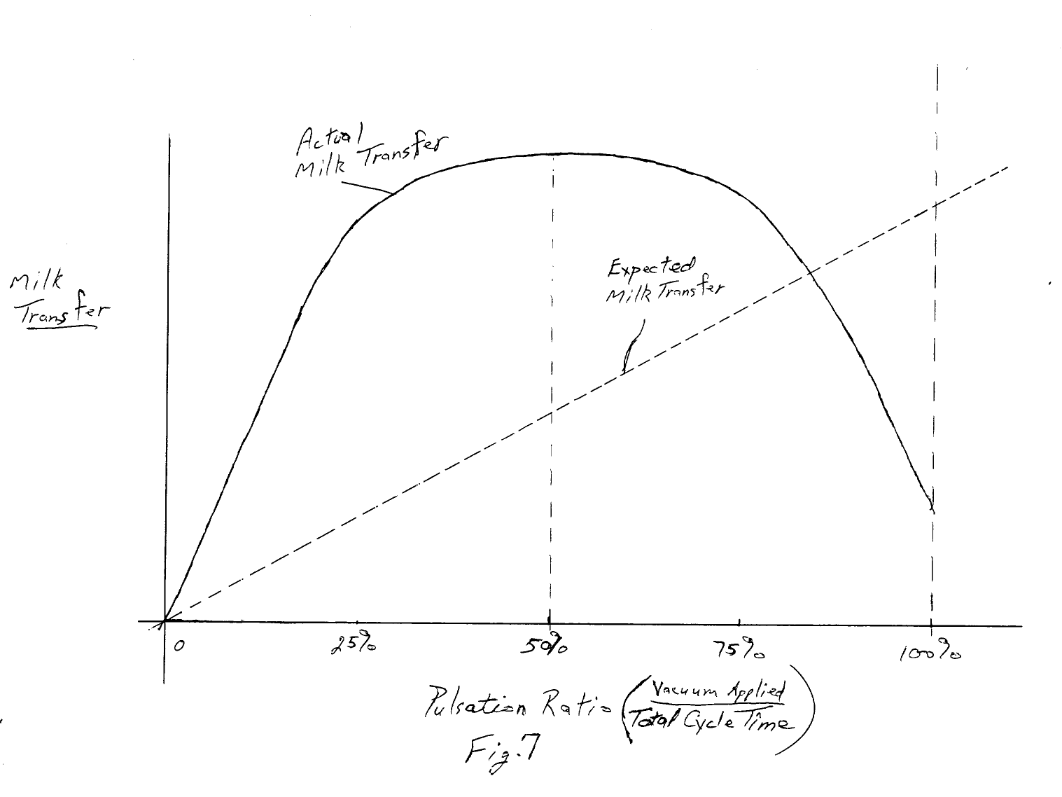

The apparatus of claim 5 can be controlled by the control system shown in FIG. 6. The control system can include a CPU 70 or the like, programmed by instructions stored in a ROM 62 and loaded in a RAM 74, if desired. Various operational parameters can be adjusted by the user as desired. For example, a pulsation ratio adjustment 76 can be provided, as well as a vacuum adjustment 78. The pulsation ratio can be adjusted at several preset values between about 20% and about 80% (vacuum applied/total cycle time), and the pulsation rate can be adjusted at several present values between about 41 and 65 pulsation cycles per minute. The ratio and rate could also be continuously variable, if desired.

Using the parameters set by the user, the CPU 70 controls the valves 62, 64, through a de-multiplexer 80 and valve drivers 82. The vacuum created by the generator 50 can be controlled through a driver 84. Of course, other control systems could be used instead of this system.

Each pulsation cycle applies vacuum to the breast for a portion of the cycle, and substantially cuts off the vacuum during the remaining portion of each cycle. The proportion of time the vacuum is applied divided by the total cycle time can be expressed as a percentage, as described in FIG. 7. Pulsation ratio is the comparison of time spent when the liner is moving towards and in the first position (open) versus the liner moving towards and in the second position (closed). While opening the liner longer would be expected to increase the rate of transfer of milk, the rate of transfer tends to decrease and the sensation of pain increases as the amount of time vacuum is applied is increased to 100%.

The present inventors have discovered that while production initially increases, milk production actually decreases as the ratio approaches 100% vacuum. While milk production varies somewhat from individual to individual and from time to time, the present inventors believe that a pulsation ratio between about 20% and about 80% vacuum (vacuum exposure/total cycle time) will produce the best results using pulsation.

The valve controller of FIG. 6 can be variable, if desired, so that the user can adjust the pulsation ratio and/or rate to obtain optimum milk transfer with the least amount of discomfort. To achieve a desired % or time open to vacuum in an open-close cycle, or pulsation cycle, the valve controller uses time control to open and close valves 62, 64 (FIG. 5). The valve controller can also control vacuum generation to produce pulsations or other varying increase and decrease of system vacuum, and thereby vary the inner chamber vacuum, if the vacuum reservoir 52 is not used. The inner chamber vacuum could also be modulated using proportional valves in the lines 26, with appropriate control, in which case the reservoir can be used, if desired.

The valve controller can be microprocessor driven, as described, or it can be a simple mechanical device. It is also contemplated that a stimulation cycle can be programmed to be used at the beginning of each milk expression session to increase sensitivity, blood flow and ultimately, milk production. The duration of the stimulation cycle along with the speed and vacuum levels can be user adjustable.

The many advantages of this invention are now apparent. The pulsation ratio can be selected to optimize the milk transfer rate. Milk transfer can be optimized on an individual basis by allowing the user to select the pulsation ratio.

While the principles of the invention have been described above in connection with a specific apparatus and applications, it is to be understood that this description is made only by way of example and not as a limitation on the scope of the invention.

U.S. patent application Ser. No. 09/876,891, filed Jun. 7, 2001, is hereby incorporated by reference.

What is claimed is:

1. A method for extracting milk from a human breast comprising the steps of:
placing the breast in a cup having a vacuum port, the cup also having a flexible liner that allows milk to be extracted from the breast by moving the liner in pulsation cycles between a first, open position, and a second, closed position, the milk being drawn into the vacuum port by the vacuum when the liner is in the first position, the liner pressing against at least part of the breast enough to substantially reduce the flow of milk from the breast in the second position;
applying a vacuum to the vacuum port; and
releasing and compressing the liner in a pulsating manner at a predetermined pulsation ratio of time in the first position divided by the total time of the pulsation cycle that produces acceptable milk transfer and comfort.

2. The method of claim 1 wherein the vacuum is applied to the breast between about 20% and about 80% of each pulsation cycle by releasing the liner, and milk flow is substantially cut off for the remaining part of the cycle by compressing the liner against the breast.

3. The method of claim 1, wherein the vacuum is generated using a pump to generate the vacuum, the pulsation being generated by pulsation valves controlled by switching circuits.

4. The method of claim 3, wherein the pulsation ratio is variable and is adjustable by the user.

5. The method of claim 3 wherein the pump generates a substantially constant vacuum.

6. The method of claim 3, wherein the pump generates a varying vacuum.

7. Apparatus for extracting milk from a human breast comprising:
at least one breast cup having a vacuum port and a flexible liner;
a vacuum source connected to the vacuum port;
a pulsated air source capable of opening and closing the liner; and
a valve controller connected to atmosphere and/or vacuum, the valve controller generating pulsation cycles such that the liner allows vacuum to reach the breast through the vacuum port at a preset or user determined pulsation ratio that produces acceptable milk transfer and comfort.

8. The apparatus of claim 7 wherein the vacuum is applied to the breast between about 20% and about 80% of the pulsation cycle and then presses against at least part of the breast to reduce the vacuum during the remaining portion of the pulsation cycle.

9. The apparatus of claim 7 wherein the vacuum is generated using a pump to generate the vacuum, the pulsations being generated by pulsator valves controlled by switching circuits.

10. The apparatus of claim 7 wherein the pulsation ratio is variable and can be adjustable (adjusted) by the user.

11. The apparatus of claim 7 wherein the pump generates a substantially constant vacuum.

12. The apparatus of claim 7 wherein the pump generates a varying vacuum.

13. The apparatus for extracting milk from a human breast having a nipple, comprising:
at least one breast cup having dual chambers;
and a flexible liner between the chambers;
each chamber being connected to a vacuum source, alternately vented to atmospheric conditions, thereby controlling the position of the liner to (a) open to extract milk from the breast, and (b) compress the liner against the breast, and
valve controllers connected to the vacuum source and atmosphere, the valve controllers generating pulsation cycles such that the liner allows vacuum to reach the breast through the vacuum port between 20% and 80% of the pulsation cycle, and presses the liner against at least part of the breast to substantially reduce the vacuum applied to the breast during the remaining portion of the pulsation cycle.
